(19) United States
(12) Patent Application Publication
(10) Pub. No.: US 2007/0282295 A1
(43) Pub. Date: Dec. 6, 2007

(54) RECEPTACLE FOR DELIVERYING A MEDIUM

(75) Inventor: Michael Horn, Oldendorf (DE)

Correspondence Address: VIDAS, ARRETT & STEINKRAUS, P.A., SUITE 400, 6640 SHADY OAK ROAD, EDEN PRAIRIE, MN, 55344, US

(73) Assignee: LINDAL Ventil GmbH, Bad Oldesloe (DE)

(21) Appl. No.: 11/756,268

(22) Filed: May 31, 2007

(30) Foreign Application Priority Data
Jun. 1, 2006 (DE) 10 2006 026 382.0
Jun. 17, 2006 (DE) 10 2006 027 911.5

(51) Int. Cl. A61B 19/00

(52) U.S. Cl. 604/408; 604/403

(57) ABSTRACT

Receptacle for delivering a medium, which is located in a bag and is delivered via a pressurized propellant; the receptacle has a receptacle body with a first opening, which has a circular edge and is closed with a valve cover, characterized in that the bag has an opening with a circular edge, which is connected with the circular edge of the receptacle body.

### CROSS-REFERENCE TO RELATED APPLICATIONS

Not applicable.

### STATEMENT REGARDING FEDERALLY SPONSORED RESEARCH

Not applicable.

### BACKGROUND OF THE INVENTION

The present invention relates to a receptacle for delivering a medium, which is located in a bag.

For different media, for example for paste-like, gel-like media or foamy media, it is known to put the medium to be delivered in a bag and to deliver it via a propellant, which is located outside the bag. The propellant is pressurized and the bag is opened via a valve so that the propellant pushes the medium out of the bag. For this, it is required that the bag be sealed off from the valve, so that the pressurized propellant only pushes the medium out of the bag into the valve and the medium cannot leak into the space between the bag and the receptacle.

Different embodiments of valves, on the valve bodies of which the bag for the medium is attached, are known and the technical term for them is Bag-on-Valve. Special technical requirements for such valves with bags result with respect to the impermeability of the bag attachment to the valve body.

### BRIEF SUMMARY OF THE INVENTION

The receptacle according to the invention serves to deliver a medium from a bag. The delivery takes place via a pressurized propellant, which pushes on the bag. The receptacle has a receptacle body with a first opening, which has a circular edge and is closed with a valve cover. The valve cover covers the first opening of the receptacle body and has for example a central opening, into which the valve can be inserted. In the case of the receptacle according to the invention, the bag has an opening with a circular edge, which is connected with the circular edge of the receptacle body. The bag fastened according to the invention, which receives the medium to be delivered, is hung in the receptacle body and is connected with its edge with the circular edge of the receptacle body. In this embodiment, the bag is fastened in the receptacle body on its upper edge. A special advantage of the bag system according to the invention is that the sealing problems between the bag and the valve are resolved. According to the invention, the bag is connected with the receptacle body, whereby a sealed connection can be achieved in a simple manner.

In a preferred embodiment, the bag is designed as a tube, which is closed on one end. In one option, the tube is fused on one end and thus closed. Conventionally, bags for bag valves were made of two flat film sheets lying on top of each other, which were fused together along their perimeter. With respect to the solution according to the invention with a tube bag, a bag manufactured in this manner has the disadvantage that leaks can occur on the fused lateral edges.

In another option, the bag is closed with a ring-shaped element. Depending on the type of clamp, the ring-shaped element holds together a constricted tube end. The end of the bag is preferably clamped together by the ring-shaped element. Advantageously, the ring-shaped element provided as the clamp is at least partially made of aluminum or an aluminum allow and is pressed on the constricted bag end during fitting.

In a preferred embodiment, the diameter of the tube is approximately equal to the diameter of the bag body. In order to connect the bag and the receptacle body, the bag in its edge area is fused with the edge area of the receptacle body. Various heat-sealing technique can hereby be used. Furthermore, the bag in its edge area can be connected with the edge area of the receptacle body using adhesive, in particular a hot-melt adhesive. In another possible embodiment, the edge area of the bag is fused with the edge area of the receptacle body using ultrasound. Depending on the fastening type selected, the receptacle body is suitably coated on the inside, in particular in its edge area in order to create a connection with the material of the bag.

In a preferred further embodiment of the receptacle according to the invention, the valve cover is crimped with the valve body. The edge area of the bag connected with the receptacle body is hereby clamped between the edge of the receptacle body and the edge of the valve cover.

In a preferred embodiment, a delivery valve is inserted into the valve cover. The type of valve us thereby adjusted based on the medium to be delivered.

In a preferred embodiment, a second opening is provided in the receptacle body, which is closed by a plug. The valve cover is at least partially covered on its inside with a protective layer. The protective layer can be polyethylene terephthalate (PET), polypropylene (PP) and/or a suitable protective paint.

### BRIEF DESCRIPTION OF THE DRAWINGS

The receptacle according to the invention is described in greater detail below using an example.

FIG. 1 shows a cut through the receptacle according to the invention with an unattached valve cover.

FIG. 2 shows a detailed view of the edge area from FIG. 1,

FIG. 3 shows a cut through the receptacle according to the invention with an attached valve cover,

FIG. 4 shows a detailed view of the edge area when the valve covered is attached.

### DETAILED DESCRIPTION OF THE INVENTION

While this invention may be embodied in many different forms, there are described in detail herein a specific preferred embodiment of the invention. This description is an exemplification of the principles of the invention and is not intended to limit the invention to the particular embodiment illustrated

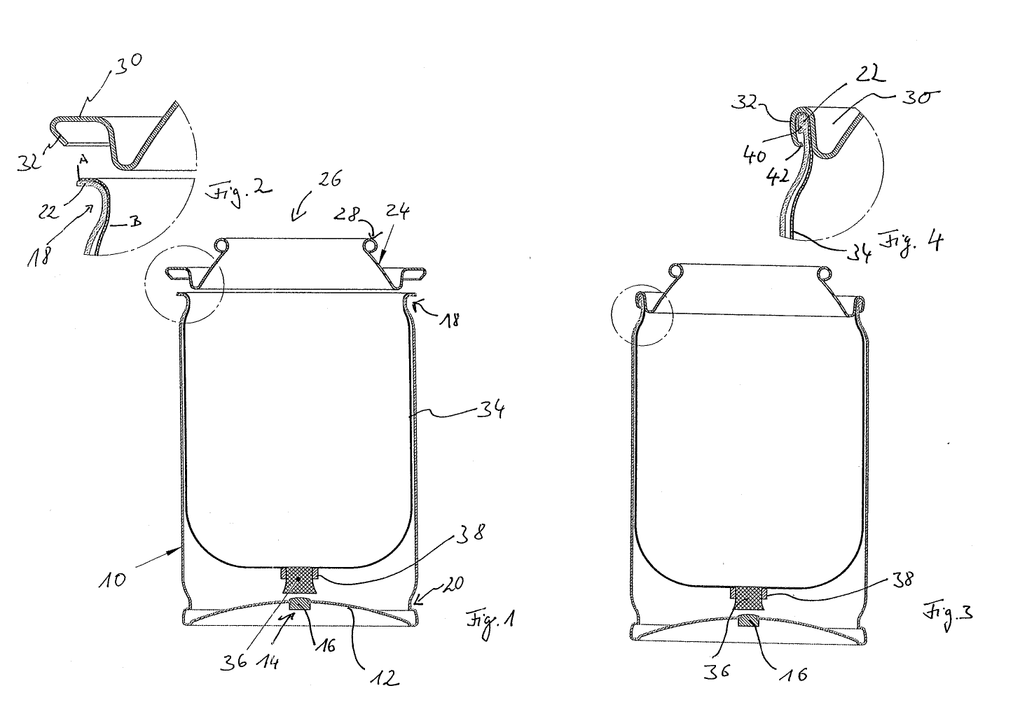

FIG. 1 shows a mainly cylindrical receptacle 10 with an inwards-curved bottom 12, which has a central opening 14. The opening 14 is pressurized by a plug 16. The side wall of the receptacle body 10 extends mainly cylindrically. The receptacle wall is only curved inwards in an upper and lower section 18. In the area of the upper tapering, the end of the side wall sticks out from the circular flange 22 approximately at a 90° angle. A dome-shaped valve cover 24 with a central intake opening 26 is provided for fastening on the receptacle body 10. A valve suitable for the medium, for example a tilt valve, is inserted into the intake opening 26 and is held in the valve cover 24 by the side wall 28. The valve cover 24 has an outer circular fastening flange 30 with a beveled, inwards sloping web 32.

A tube-shaped bag 34 is hung in the receptacle body 10, the central area of which is designed cylindrically and rests against the inside wall of the receptacle body 10. The bag 34 is constricted in its lower section. The constriction is central in the tube and closes it, in order to obtain a bag 34 closed on the bottom end. The constricted bag material hereby forms an appendage 36. The appendage 36 is held together by a clamp 38, which is crimped on the appendage 36. The clamp 38 can be made of various materials; it can e.g. be made of aluminum, which is deformed for the closing of the appendage 36. Depending on the wall strength and the material of the bag 34, the appendage 36 can also be sealed, for example by a sealing compound inserted from the inside or outside.

On the opposite-lying end, the tube-shaped bag 34 is connected with the edge area of the receptacle body 10. As can be seen in FIG. 2, the outside of the bag 34 rests against the inside wall of the receptacle in an area between drawn-in lines A and B. In the area between A and B, the bag material 34 is connected with the inside wall of the receptacle body. For example, hot-melt adhesive, a heat-sealing process or ultrasound fusing can be used. Combinations of these or other connection types are also generally possible. The connection area A to B hereby extends via the flange 22 that sticks out horizontally and partially via the inside wall of the receptacle body 10 in the area of the constriction 18. Particularly advantageous in the case of this connection type is that the bag can be fastened in a sealed manner on the receptacle body for a tube-shaped bag, the diameter of which is approximately equal to the diameter of the receptacle body, without drape and thus without potential leak spots. The valve cover 24 is placed on the fastened bag and is fastened with the cylinder body through the bending of flange 30 and web 32. For the fastening, the flange 22 that sticks out from the receptacle body is bent by approx. 90°, wherein the outer area 40 of flange 22 is angled by 180°. The bag material extends along the inside wall of the receptacle body and thereby follows the 180° turn of the wall. The connection is surrounded by the flange 30 and the web 32 of the valve cover 24. The web 32 covers the wall of the receptacle body including the bag material from the outside. The flange 30 forms the inner contact for the bent side wall. The outer edge of the web 32 is bent inwards and forms a hook 42.

In the exemplary embodiment shown, the valve cover 24 is placed directly on the receptacle body with the inserted tube bag. The transition from the inside wall of the bag to the valve cover can also be sealed by an additional seal, just like the appendage 36 can be sealed by additional material. This type of seal can e.g. be arranged between the valve cover and the bag in the area of the bent inside wall 22, 40 in the form of a sealing ring.

As an alternative to the shown closure of the bag via a clamp, the bag hung in the inside of the receptacle can also be fused on its bottom end. This only creates one fused seam in comparison to the known solutions, which have a circumferential fused seam. Furthermore, the valve cover 24 is coated on the inside with polyethylene terephthalate (PET), polypropylene (PP) or paint. Polyethylene (PE), oriented polypropylene films (OPP) and PE-tubes coated with aluminum have proven to be especially suitable bag materials. Metallized, oriented polypropylene film can also be used.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

What is claimed is:

1. Receptacle for delivering a medium, which is located in a bag (34) and is delivered via a pressurized propellant; the receptacle has a receptacle body (10) with a first opening, which has a circular edge (22, 18) and is closed with a valve cover (24), characterized in that the bag has an opening with a circular edge, which is connected with the circular edge of the receptacle body.

2. Receptacle according to claim 1, characterized in that the receptacle is designed as a tube, which is closed on one end.

3. Receptacle according to claim 2, characterized in that the bag is fused on its closed end.

4. Receptacle according to claim 2, characterized in that the bag is closed on its closed end with a ring-shaped element.

5. Receptacle according to claim 4, characterized in that the element is clamped on one end of the bag.

6. Receptacle according to claim 4, characterized in that the ring-shaped element is made at least partially of aluminum.

7. Receptacle according to claim 1, characterized in that the diameter of the tube is approximately the same as the diameter of the receptacle body.

8. Receptacle according to claim 1, characterized in that the bag in its edge area is fused with the edge area of the receptacle body.

9. Receptacle according to claim 1, characterized in that the bag in its edge area is fused with the edge area of the receptacle body via an adhesive.

10. Receptacle according to claim 1, characterized in that the bag in its edge area is sound-fused with the edge area of the receptacle body via ultrasound.

11. Receptacle according to claim 1, characterized in that the valve cover is crimped with the receptacle body.

12. Receptacle body according to claim 1, characterized in that a delivery valve is inserted in the valve cover.

13. Receptacle according to claim 1, characterized in that a second opening is provided in the receptacle body, which is closed by a plug.

14. Receptacle according to claim 1, characterized in that the valve cover is at least partially covered with a protective layer.

15. Receptacle according to claim 14, characterized in that the protective layer has polyethylene terephthalate and/or polypropylene and/or a protective paint.
